(19) United States
(12) Patent Application Publication
(10) Pub. No.: US 2016/0066836 A1
(43) Pub. Date: Mar. 10, 2016

(54) GAMIFIED ELECTROMYOGRAPHIC NEUROMUSCULAR REEDUCATION THERAPY SYSTEM

(71) Applicant: Maria E. Loveland Schneider, Carrollton, TX (US)

(72) Inventor: Maria E. Loveland Schneider, Carrollton, TX (US)

(73) Assignee: DYNOFIT, INC., Los Alamos, NM (US)

(21) Appl. No.: 14/848,794

(22) Filed: Sep. 9, 2015

Related U.S. Application Data
(60) Provisional application No. 62/047,658, filed on Sep. 9, 2014.

(51) Int. Cl. A61B 5/00; A61B 5/04; A61B 5/0492

(52) U.S. Cl. CPC A61B 5/486; A61B 5/0492; A61B 5/04012; A61B 5/4836; A61B 5/742; A61B 5/7475; A61B 5/6898; A61B 5/0004; A61B 5/0022; A61B 2505/09

(57) ABSTRACT

A method, system and apparatus for gamified electromyographic neuromuscular reeducation therapy may include an EMG receiver and a mobile computing device. In some embodiments, the mobile computing device receives an EMG signal from an EMG sensor couplable to a muscle of a user. The mobile computing device converting the EMG signal to an action item of a game feature in an electronic game. In some embodiments, the electronic game includes secondary features displayed on a user interface and adapted to instigate a user to perform therapeutic movements associated with a neuromuscular reeducation therapy. In some embodiment, the mobile computing device executes the action item of the game feature and displays a result of the action item on the user interface.

### STATEMENT REGARDING FEDERALLY SPONSORED RESEARCH OR DEVELOPMENT

This invention was made in part with government support under 1R43HD080234-01 awarded by the National Institute of Health. The government has certain rights in the invention.

### CROSS-REFERENCE TO RELATED APPLICATIONS

Pursuant to 35 U.S.C. §119(e), this application claims priority from, and hereby incorporates by reference for all purposes, U.S. Provisional Patent Application Ser. No. 62/047,658, entitled “GAMIFIED ELECTROMYOGRAPHIC NEUROMUSCULAR REEDUCATION THERAPY SYSTEM,” filed Sep. 9, 2014.

### BACKGROUND OF THE DISCLOSURE

Eight million people in the U.S. suffer from strokes, cerebral palsy, and spinal cord injuries—all of which can result in a patient's loss of the ability to properly control his/her muscles. A variety of other neurological conditions can also result in this loss of ability.

Motor skill issues can be addressed through neuro-muscular re-education therapies (NMRT) to dramatically improve a person's quality of life (e.g. a person might re-learn to walk). The currently available technology for performing electromyography (EMG) based NMRT usually connects a dedicated computer of some sort to the patient with cables. This is unwieldy, expensive, and generally limited to a clinical setting under the direct supervision of a Physical Therapist or Occupational Therapist. The therapy is usually accordingly expensive, and is also usually boring in its current form. The resulting lack of engagement due to boredom is problematic because it can make adherence a problem, even if the therapy can be sent home at a lower cost.

### SUMMARY

In some embodiments, the system includes an electromyography sensor (EMG) worn by a user that is connected wirelessly to a form of mobile device or other computer. EMG data representing neural activation potentials are sent from the sensor to the mobile device. The mobile device processes the EMG data, and the results of that processing are used to control the behavior of software running on the mobile device. This software displays outputs to the user, allowing, for instance, a videogame to be played tailored to accomplish a specific form of therapy.

In a first aspect, there is provided a method, implemented in computer-executable program(s) or computer-executable instruction(s) in computer system(s) or device(s) that includes the steps of receiving an EMG signal from an EMG sensor couplable to a muscle of a user; converting the EMG signal to an action item of a game feature in an electronic game, wherein the electronic game includes secondary features displayed on a user interface and adapted to instigate a user to perform therapeutic movements associated with a neuromuscular reeducation therapy; executing the action item of the game feature; and displaying a result of the action item on the user interface, wherein the result includes a comparison of the action item to the secondary feature. In some embodiments, for example, the secondary feature may be a crevasse or space that the user must move a ball over in order to continue playing the game. The user moves the ball by flexing the user's muscle. The amplitude of the movement of the ball may correspond to the amplitude of the flexing of the user's muscle.

In some embodiments, executing the action item includes moving the game feature between a first location and a second location. For example, in some embodiments, the game feature is a flying machine that moves from a first location (for example, a low position on the user interface) to a second location (for example, a high position on the user interface) based on the EMG signal received from the user's muscle.

In another embodiment, a characteristic of the secondary feature corresponds to the movement associated with the neuromuscular reeducation therapy. For example, in some embodiments the second feature is an obstacle over which the game feature must jump in order to proceed with the game. In some embodiments, the height of the secondary feature corresponds to the amplitude of flexing required by the neuromuscular reeducation therapy.

In yet another embodiment, the action item of the game feature corresponds in magnitude to the EMG signal. For example, in some embodiments the height to which the game feature moves corresponds to the EMG signal received from the user and the flexing (or relaxing) of the user's muscle.

In still another embodiments, the secondary feature corresponds in magnitude to a desired EMG signal.

In yet another embodiment, the secondary feature includes a plurality of secondary features, wherein the frequency of the plurality of secondary features corresponds to a desired frequency of EMG signals. For example, in some embodiments a user's therapy requires rapid flexing and releasing of the user's muscle. As such, the secondary features may be spaced closely together such that the user must flex (and release) the user's muscle at the desired frequency to avoid the secondary features in the game.

In a second aspect, there is described a computer system for assisting a patient with neuro-muscular re-education therapy. In some embodiments, the system includes a mobile computing device, wherein the mobile computing device includes a user interface and a game, wherein the game includes a game feature and a secondary feature, wherein the secondary feature corresponds to a desired neuro-muscular re-education therapy movement; and an EMG receiver coupleable to a patient's muscle, wherein the EMG receiver is communicatively coupled to the mobile computing device to transmit an EMG signal to the mobile computing device. In some embodiments, the mobile computing device receives the EMG signal and modifies a characteristic of the game feature based on the EMG signal, such as, for example, the location of the game feature in the game. In some embodiments, the mobile computing device compares the modified characteristic of the game feature to the secondary feature to determine if the user's muscle performed the desired neuro-muscular re-education therapy movement. For example, in some embodiments the computing device compares the magnitude of movement of the game feature to the magnitude of the secondary feature to determine if the user's muscle flexed to the desired magnitude. In some embodiments, the user can see on a user interface whether the user flexed his or her muscle to the desired magnitude.

In some embodiments, the EMG receiver is communicatively coupled to the mobile computing device by a wireless coupling.

In yet other embodiments, the EMG receiver includes a plurality of EMG sensors, wherein the EMG sensors are coupled by a flexible coupling.

In some embodiments, the EMG receiver includes a plurality of EMG sensors, wherein the EMG sensors are coupled by a rigid coupling.

In other embodiments, the desired neuro-muscular re-education therapy movement includes flexing the user's muscle.

In still other embodiments, the desired neuro-muscular re-education therapy movement includes relaxing the user's muscle.

In some embodiments, the amplitude of the secondary feature corresponds to an amplitude of the desired neuro-muscular re-education therapy movement.

In a third aspect, there is described an apparatus for assisting a patient with neuro-muscular re-education therapy that includes a first EMG sensor including a first skin coupling portion for coupling to a user's skin at a user's muscle; a second EMG sensor including a second skin coupling portion for coupling to the user's skin at the user's muscle; a wireless transceiver for sending EMG signals received by the first and second EMG sensors to a mobile computing device that includes a game configured to assist the user with neuro-muscular re-education therapy; and a coupling between the first EMG sensor and the second EMG sensor.

In some embodiments, the apparatus further includes a battery, an LED and an On/Off switch.

In other embodiments, the apparatus further includes an analog to digital converter and signal processing electronics.

In yet another embodiment, the coupling is flexible to allow a user to position the first EMG sensor a user defined distance from the second EMG sensor. In other embodiments, the coupling is resilient and comprises a coil.

In still another embodiment, the coupling is rigid so that a distance between the first EMG sensor and the second EMG sensor is a predetermined distance.

In yet another embodiment, the apparatus includes a third EMG sensor.

### DESCRIPTION OF THE FIGURES

The accompanying drawings facilitate an understanding of the various embodiments.

FIG. 1 is a block diagram of an exemplary operating environment for use in implementing an embodiment or a portion of an embodiment of the present disclosure.

FIG. 2 is a block diagram of a network environment for use in implementing an embodiment of the present disclosure.

FIG. 3 illustrates an exemplary operating environment with a user and mobile device.

FIGS. 4A-4C illustrate an embodiment of the EMG sensor with two electrodes and variable spacing.

FIG. 5 is a schematic illustration of an embodiment of the EMG sensor electronics with two electrodes and variable spacing.

FIG. 6 is a schematic illustration of an embodiment of an embodiment of an EMG sensor with three electrodes and fixed spacing.

FIG. 7 illustrates an extension member for use in varying the location of the electrodes.

FIG. 8 is a block diagram of an embodiment of the EMG sensor electronics with three electrodes and fixed spacing.

### DETAILED DESCRIPTION

Embodiments described herein generally relate to a new apparatus, system and method for performing neuro-muscular re-education therapy using electromyography and mobile devices. Motor skill issues can be addressed through neuro-muscular re-education therapies (NMRT) to dramatically improve a person's quality of life (e.g. a person might re-learn to walk). However, NMRT is often expensive, must be supervised by a trained operator at a dedicated location, such as a doctor's office, and is not entertaining, thus reducing the rate at which patient actually perform assigned therapy. The embodiments described herein provide an inexpensive and, in some embodiment, mobile system, apparatus and method for performing electromyography (EMG) based NMRT through the use of mobile application games that are configured to address a particular neuro-muscular re-education therapy. The mobile application games provide an engaging environment in which patients perform NMRT to increase patient performance of prescribed NMRT and to increase therapeutic efficiency.

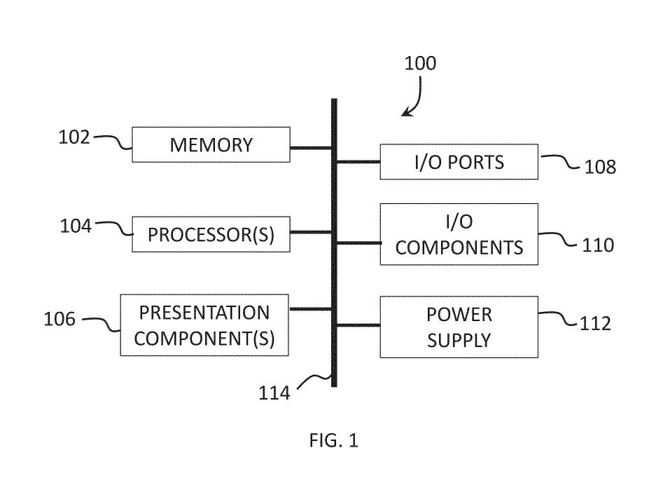

Having described a general overview of the embodiments described herein, an exemplary operating environment is described below. Referring initially to FIG. 1 in particular, an exemplary operating environment for implementing the present invention is shown and designated generally as computing device 100. The computing device 100 is but one example of a suitable computing environment and is not intended to suggest any limitations as to the scope of use or functionality of the disclosure. Neither should computing device 100 be interpreted as necessarily having any dependency or requirement relating to any one or combination of components illustrated. The disclosure may be described in the general context of a computer code or machine-usable instructions, including computer-executable instructions such as program modules, being executed by a computer or other machine, such as a PDA, smartphone or other handheld device. Generally, program modules that include routines, programs, objects, components, smart-phone applications, data structures, and the like, refer to code that perform particular tasks or implement particular abstract data types. The modules described herein may represent executable source code written in a well-known language, such as, for example, C, C++, C#, Java, or the like. Embodiments described herein may be practiced in distributed computing environments where tasks are performed by remote-processing devices that are linked through one or more communications networks.

With continued reference to FIG. 1, computing device 100, which may be a mobile smart phone or other computing device, includes a bus 114 that directly or indirectly couples the following devices: memory 102, one or more processors 104, one or more presentation components 106, input/output ports 108, input/output components 110 and an illustrative power supply 112. Bus 114 represents what may be one or more busses (such as an address bus, data bus, or combination thereof). Although the various blocks of FIG. 1 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. It will be understood by those skilled in the art that such is the nature of the art, and, as previously mentioned, the diagram of FIG. 1 is merely illustrative of an exemplary computing device that can be used in connection with one or more embodiments of the present invention. Distinction is not made between such categories as “workstation,” “server,” “laptop,” “hand-held device,” “smartphone,” etc., as all are contemplated within the scope of FIG. 1 and reference to as a “computing device.”

The computing device 100 typically includes a variety of computer-readable media. By way of example, and not limitation, computer-readable media may comprise Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices, carrier wave or any other medium that can be used to encode desired information and be accessed by the computing device 100.

The memory 102 includes computer-storage media in the form of volatile and/or nonvolatile memory. The memory 102 may be removable, nonremovable, or a combination thereof. Exemplary hardware devices include solid-state memory, hard drives, cache, optical-disc drives, etc. The computing device 100 includes one or more processors 104 that read data from various entities such as memory 102 or I/O components 110. The presentation component(s) 106 present data indications to a user or other device. Exemplary presentation components 106 include a display device, speaker, printing component, vibrating component, etc.

The I/O ports 110 allow computing device 100 to be logically coupled to other devices including I/O components 110, some of which may be built in. Illustrative components include a microphone, EMG sensors, wireless transceivers, joystick, game pad, satellite dish, scanner, printer, wireless device, keypad etc.

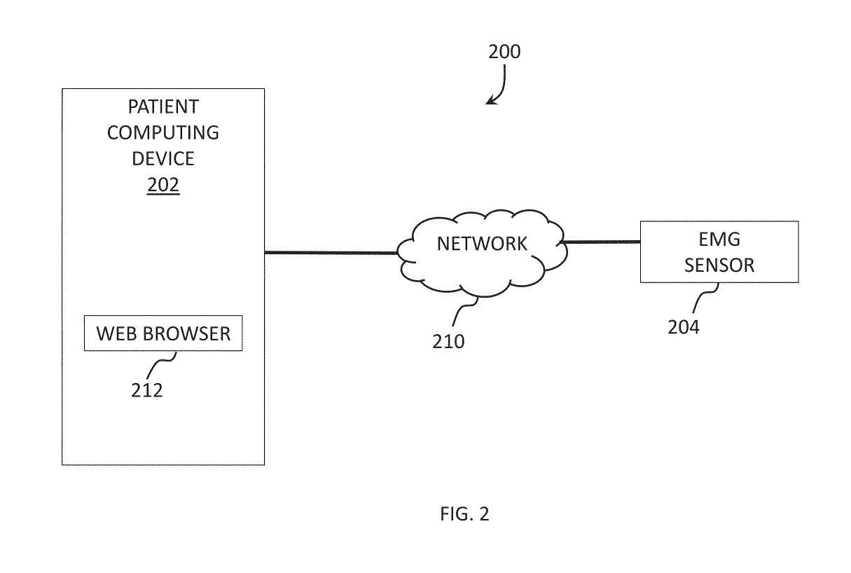

Turning now to FIG. 2, a block diagram depicting a networking architecture 200 is shown for use in implementing embodiments described herein. The networking architecture 200 includes a patient computing device 202 and an EMG sensor 204, which may communicate with each other via network 210. The networking architecture 200 is merely an example of one suitable networking environment and is not intended to suggest any limitation as to the scope of use or functionality of the present invention. Neither should networking architecture 200 be interpreted as necessarily having any dependency or requirement related to any single component or combination of components illustrated therein.

The client computing device 202 may be any type of computing device, such as device 100 described above with reference to FIG. 1. By way of example only and not limitation, the client computing device 202 may be a personal computer, desktop computer, laptop computer, handheld device, cellular phone, digital phone, smartphone, PDA, or the like. It should be noted that embodiments are not limited to implementation on such computing devices.

The network 210 may include any computer network or combination thereof. Examples of computer networks configurable to operate as network 210 include, without limitation, a wireless network, landline, cable line, digital subscriber line (DSL), fiber-optic line, local area network (LAN), wide area network (WAN), metropolitan area network (MAN), Bluetooth connection, or the like. The network 210 is not limited, however, to connections coupling separate computer units. Rather, the network 210 may also include subsystems that transfer data between servers or computing devices. For example, the network 210 may also include a point-to-point connection, the Internet, an Ethernet, an electrical bus, a neural network, or other internal system.

In an embodiment where the network 210 comprises a LAN networking environment, components may be connected to the LAN through a network interface or adapter. In an embodiment where the network 210 comprises a WAN networking environment, components may use a modem, or other means for establishing communications over the WAN, to communicate. In embodiments where the network 210 comprises a MAN networking environment, components may be connected to the MAN using wireless interfaces or optical fiber connections. Such networking environments are commonplace in offices, enterprise-wide computer networks, intranets, and the Internet. It will be appreciated that the network connections shown are exemplary and other means of establishing a communications link between the computers may also be used.

Furthermore, the network 210 may also include various components necessary to facilitate communication with a mobile phone (e.g., cellular phone, Smartphone, Blackberry®). Such components may include, without limitation, switching stations, cell sites, Public Switched Telephone Network interconnections, hybrid fiber coaxial cables, or the like.

It will be understood by those of ordinary skill in the art that networking architecture 200 is merely exemplary. While the components of the networking architecture 200 are illustrated as single boxes, one skilled in the art will appreciate that they may be scalable. For example, the patient computing device 202 may in actuality include multiple boxes in communication. For example, in some embodiments, multiple users may participate in the same game to perform similar (or different) types of therapy treatments. The single unit depictions are meant for clarity, not to limit the scope of embodiments in any form.

In some embodiments, the patient computing device 202 comprises a web browser 212 which is a software application enabling a user to display and interact with information located on a web page. The web browser 212 may locate web pages by sending a transferring protocol and the URL. The web browser 212 may use various URL types and protocols, such as hypertext transfer protocol (HTTP), file transfer protocol (FTP), real-time streaming protocol (RTSP), etc. The web browsers 212 may also understand a number of file formats—such as HTML, graphics interchange format (GIF), tagged image file format (TIFF), portable document format (PDF), or joint photographic experts group (PDF) file format, and the like—the wealth of which can be extended by downloaded plug-ins. Additionally, the web browser 212 may be any browser capable of navigating the Web, such as Internet Explorer®, Netscape Navigator, Mozilla, Firefox, etc.

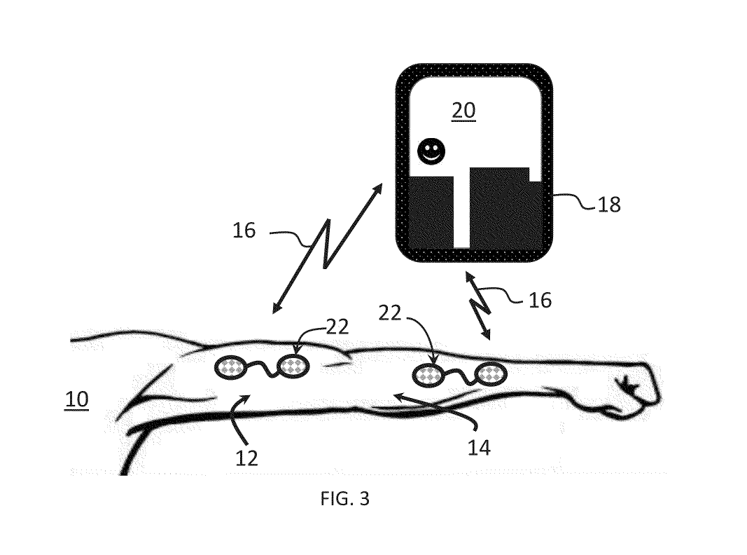

Having described a general overview of the embodiments described herein, an exemplary operating environment is described below. Referring initially to FIG. 3 in particular, an exemplary operating environment is shown. A user 10 is shown in FIG. 1, wearing EMG sensors 22 on both the bicep muscle 12 and the forearm muscle 14. The EMG sensors 22 sense the electrical signals emitted under the skin by the nerves when the muscles 12 and 14 are activated. These signals are sent by wireless radio signal 16, such as BlueTooth signal, to a computer 18, which may be similar to computing device 100. In some embodiments, the computer 18 has videogame software 20 running on it. In some embodiments, the videogame software 20 is thereby controlled by user 10′s muscle activations as sensed by the EMG sensors 22. It should be noted that while computer 18 is exemplified here as a mobile device such as a smartphone or tablet, it could be any of a number of portable devices with processing and display capability, including virtual reality glasses.

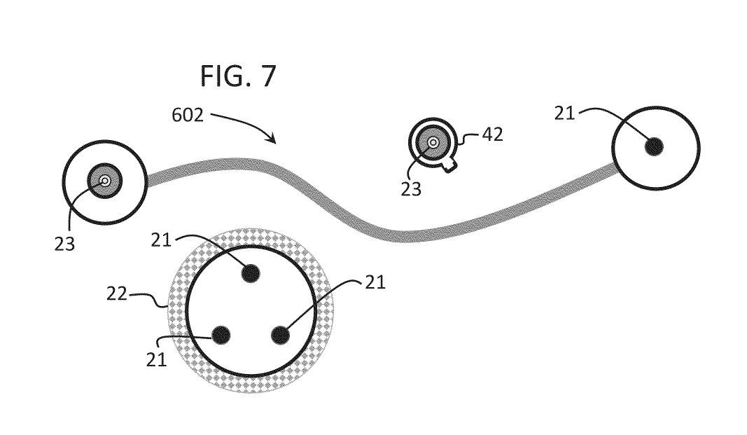

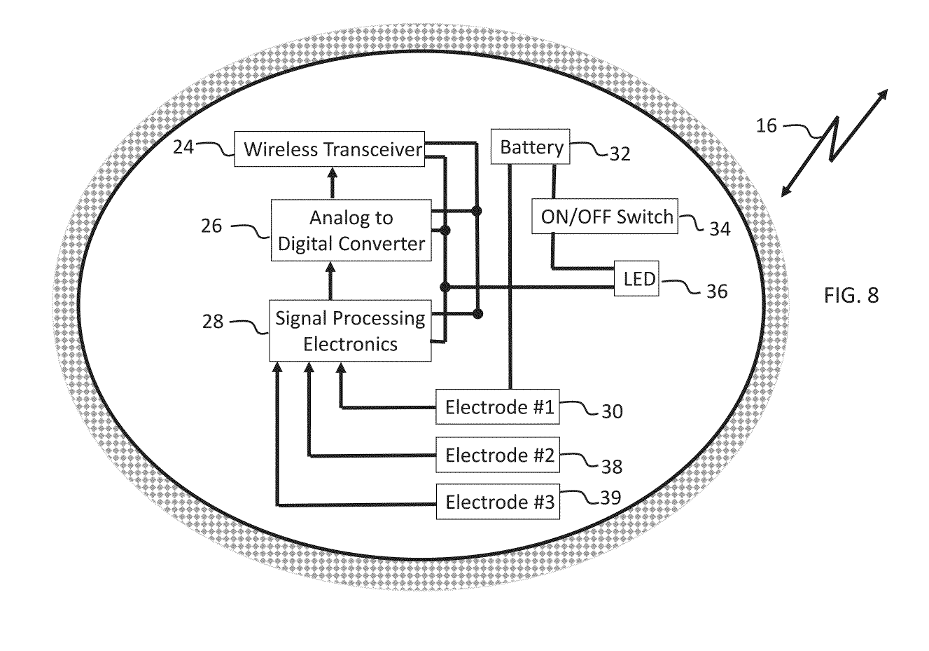

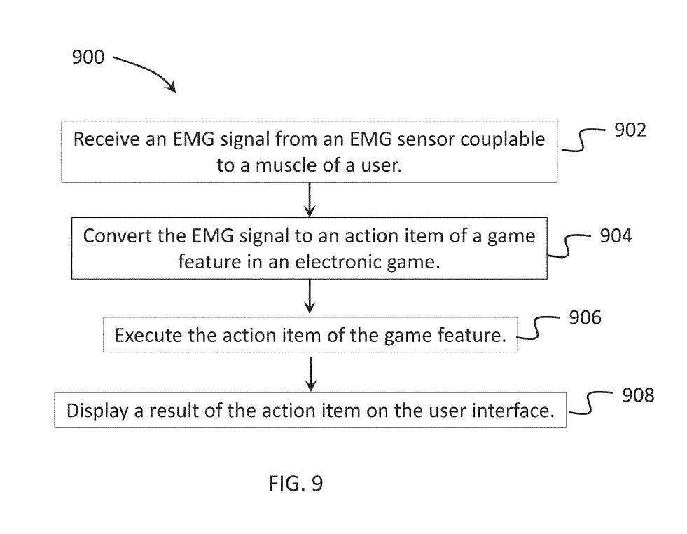

It should also be noted that wireless transceiver 24 might be based on any of a range of radio frequency technologies, such as Bluetooth, 2.4 GHz, or it might be optical or infrared. Likewise, the signal processing electronics 28 (FIG. 7) could vary widely in their actual components, as could analog to digital converter 26 (FIG. 7).

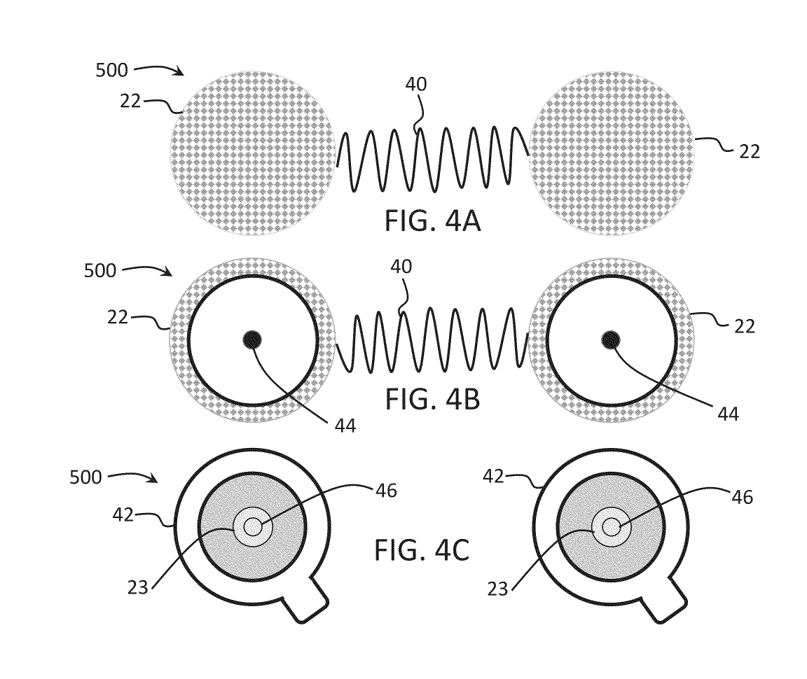

Detailed top and bottom views of an EMG sensor system 500 including EMG sensors 22 are shown in FIGS. 4A-4C. FIG. 4A illustrates a top view of the EMG sensors 22, with a flexible cable 40 connecting the two EMG sensors 22. The flexible cable 40 allows the user to position the EMG sensors 22 a desired distance from each other to obtain optimal EMG sensor readings from the user's muscle. For example, in some embodiments a user desires to receive a reading from a larger muscle, such as a quadriceps, and the flexible cable 40 allows the user to position the EMG sensors 22 in a spaced apart relationship (for example, eight to ten inches apart from each other) on the quadriceps muscle. In other embodiments, the user may position the EMG sensors 22 on the user's bicep muscle 12 so that the EMG sensors 22 are more closely spaced together, such as, for example, approximately two to six inches apart from each other.

FIG. 4B illustrates the bottom/underside of EMG sensors 22, which snap onto disposable electrode pads 42 (FIG. 4C) that are adhered to user 10's skin. As such, in some embodiments, the bottom/underside of EMG sensors 22 include a snap feature 21 that is couplable to a corresponding snap feature 23 on the disposable electrode pads 42 (FIG. 4C).

FIG. 4C illustrates adhesive electrode pads 42 that are coupleable to the EMG sensors 22 of FIGS. 4A and 4B. Electrode pad snap top 46 [NOTE TO DRAFT: Please locate item 46 in the figures] snaps into snap indentation 44 [NOTE TO DRAFT: Please locate item 44 in the figures] on the underside of EMG sensor 22.

It should be noted that an alternative embodiment the electrodes 22 may include reusable electrodes, rather than disposable pads 42. For example, in some embodiments, the reusable electrodes utilize moistening or conductive gel to achieve the appropriate coupling with the user's skin.

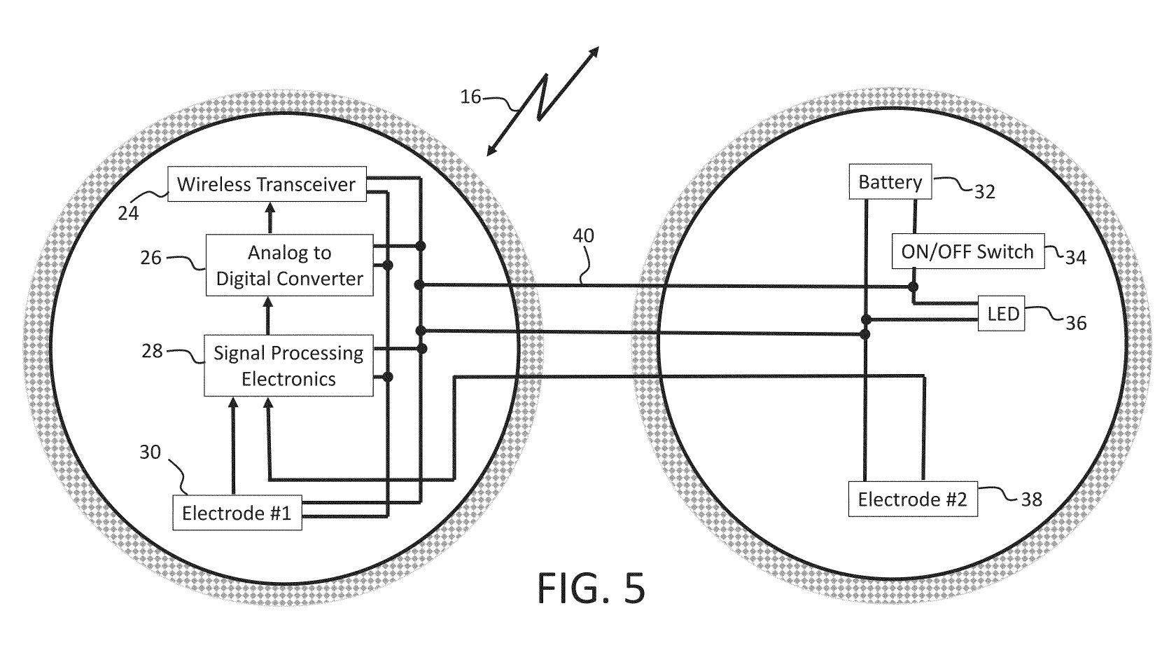

An embodiment of the components of EMG sensors 22 are shown in FIG. 5. In some embodiments, the battery 32 powers the system through ON/OFF switch 34. In other embodiments, LED 36 indicates whether or not the power is on. In some embodiments, a first electrode 30 and a second electrode 38 connect user 10′s muscle activation signals to signal processing electronics 28. The processed signal from signal processing electronics 28 may be digitized by analog to digital converter 26. In some embodiments, the digitized signal is transmitted wirelessly by wireless transceiver 24 as wireless radio signal 16 to computer 18 (FIG. 3). In some embodiments, the wireless radio signal 16 is interpreted by computer 18 to control videogame 20.

While, the embodiment of the EMG sensor 500 shown in FIGS. 2 and 3 has two electrodes 22 with variable spacing, the EMG sensor may have any number of electrodes 22 in other embodiments. For example, in some embodiments the EMG sensor has three electrodes 22 with variable spacing. In some embodiments, the three electrodes 22 are coupled together by an extendable coupling 40 (see, e.g., coupling 40 in FIGS. 4A-4C).

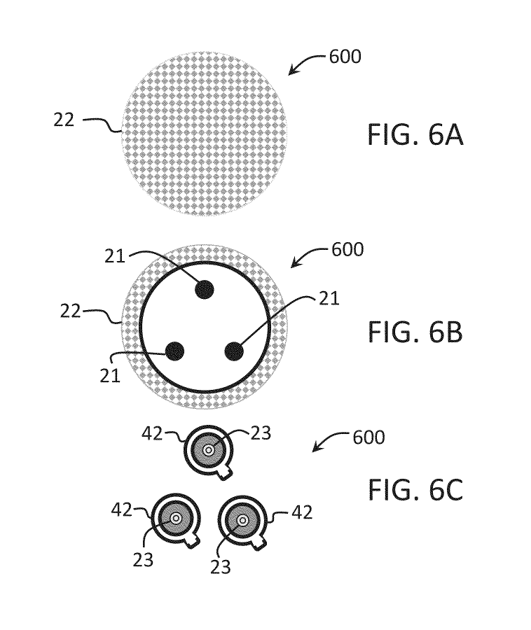

In an alternative embodiment, as illustrated in FIGS. 6A-6C, the EMG sensor system 600 includes three electrodes 22 that are fixed to each other (i.e., not variably spaced). In some embodiments, it is convenient for a physician or therapist to provide the user with fixed electrodes 22 to allow the user to more easily position all of the electrodes 22 at the appropriate locations on the user's muscle or elsewhere.

An alternative extension 602 of the EMG sensor system is illustrated in FIG. 7. The extension 602 may be used to provide viable location of the electrode 22 when coupled to an electrode 22 of the fixed embodiment (see, e.g., FIGS. 6A-6C) or the extendable embodiment (see, e.g., FIGS. 4A-4C). In some embodiments, the extension cable 602 is plugged into the EMG sensor on one end and attached to the user with a disposable electrode pad on the other end of the cable 603.

The system is operated by having user 10 adhere disposable electrode pads 42 to his/her skin above the muscles which are being targeted by the neuromuscular reeducation therapy. The EMG sensors 22 are then snapped onto the disposable electrode pads 42, one set of EMG sensors 22 for each muscles. The EMG sensors 22 are then turned on, and software is started on computer 18. The software running on computer 18 then detects EMG sensors 22 and uses the signals transmitted to control videogame 20.

Videogame 20 has a variety of potential embodiments which can be tailored to specific therapeutic goals. One example is that of a game where the objective is to jump from platform to platform without falling in between. This type of game emphasizes precise timing, without necessarily requiring a sustained contraction of the muscle controlling the game. This would be appropriate for a therapy oriented towards regaining fine control rather than strength. For example, in some embodiments the platforms are spaced such that the flexing of the user's muscles occurs at the desired frequency and timing to perform the appropriate therapy.

Videogame 20 might interpret the EMG signals as binary inputs, being either “ON” or “OFF”, or it might use the full range of the EMG input as a proportional or analog control. Thus, in some embodiments, the user's therapy may involve both amplitude of flexing the muscle and precise timing. The videogame 20 may include, for example, platforms that are spaced vertically from each other and the user must increase the amplitude of the muscle flexing to position an avatar or other game feature onto the higher altitude platforms.

A different embodiment of videogame 20 might be one where EMG signal strength is linked to maintaining a certain height above the ground to avoid crashing into obstacles. This would be appropriate for a therapy for building strength in which the muscle must be flexed at a high amplitude for an extended period of time.

In another embodiment, the videogame 20 is controlled in part by relaxation of a muscle to control an aspect of the game, e.g. where a low EMG value was required to pass under an obstacle. This would be appropriate for dealing with spasticity, where a user's muscles are involuntarily clenched.

In another embodiment of videogame 20, multiple dots are used to control different aspects of the game, or used for controlling the same aspect at different times or places in the game. For instance, walking requires a controlled, carefully timed sequence of contractions of the leg muscles. Videogame 20 might be structured in a way where that timing sequence of flexing the user's muscles is required to activate a special power within the game or to move a game piece.

Beyond NMRT therapies intended to change or heal neuronal pathways, many physical therapies are oriented towards recovery from sports or occupational injuries where repeated sessions involving numerous repetitions of a particular movement are required. An instance of videogame 20 could be used to achieve this end as well. The capability of EMG sensor 22 to monitor a very specific subset of muscles is particularly useful in this case. For example, in some embodiments, the user of the game may receive points for repeating the same muscle flexing to the same amplitude.

In some embodiments, the characteristics of the game are customized to meet the particular needs of the patient. As such, one or more of the embodiments described above may be combined together to form a game that can be used to provide NMRT to a patient with a combination of issues.

FIG. 9 illustrates a method 900 of implemented in computer-executable program(s) or computer-executable instruction(s) in computer system(s) or device(s). In some embodiments, the method 900 includes receiving an EMG signal from an EMG sensor couplable to a muscle of a user, as shown at block 902. A mobile computing device converts the EMG signal to an action item of a game feature in an electronic game, as shown at block 904. For example, in some embodiments, the action item is movement of the game feature in the electronic game between a first position and a second position. In some embodiments, the electronic game includes secondary features displayed on a user interface and adapted to instigate a user to perform therapeutic movements associated with a neuromuscular reeducation therapy. For example, in some embodiments, the secondary features are obstacles that the game feature must avoid in order to win the game. The amplitude, frequency, location, size or other characteristics of the secondary features may be configured to instigate the desired muscle movement of the user. Thus, for example, closely packed obstacles may instigate a user to flex (and relax) the user's muscle in quick succession while loosely packed obstacles may instigate a user to spend more time relaxing the user's muscles.

The method 900 may also include executing the action item of the game feature, as shown at block 906. The mobile computing device may then display a result of the action item on the user interface, as shown at block 908, wherein the result includes a comparison of the action item to the secondary feature. In some embodiments, for example, the amplitude of the user's muscle flexure is executed as movement of the game feature upwards on the game space. The mobile computing device compares the amplitude of the movement of the game feature to the amplitude of the secondary feature, which may be a wall or other obstacle, to determine if the user flexed the muscle to the desired amplitude. If so, the user passes this portion of the game. If not, the user does not pass this portion of the game.

In the foregoing description of certain embodiments, specific terminology has been resorted to for the sake of clarity. However, the disclosure is not intended to be limited to the specific terms so selected, and it is to be understood that each specific term includes other technical equivalents which operate in a similar manner to accomplish a similar technical purpose.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

In addition, the foregoing describes only some embodiments of the invention(s), and alterations, modifications, additions and/or changes can be made thereto without departing from the scope and spirit of the disclosed embodiments, the embodiments being illustrative and not restrictive.

Furthermore, invention(s) have been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention(s). Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

What is claimed is:

1. A method, implemented in computer-executable program(s) or computer-executable instruction(s) in computer system(s) or device(s), comprising steps of:
receiving an EMG signal from an EMG sensor couplable to a muscle of a user;
converting the EMG signal to an action item of a game feature in an electronic game, wherein the electronic game includes secondary features displayed on a user interface and adapted to instigate a user to perform therapeutic movements associated with a neuromuscular reeducation therapy;
executing the action item of the game feature; and
displaying a result of the action item on the user interface, wherein the result comprises a comparison of the action item to the secondary feature.

2. The method of claim 1, wherein executing the action item comprises moving the game feature between a first location and a second location.

3. The method of claim 2, wherein a characteristic of the secondary feature corresponds to the movement associated with the neuromuscular reeducation therapy.

4. The method of claim 1, wherein the action item of the game feature corresponds in magnitude to the EMG signal.

5. The method of claim 1, wherein the secondary feature corresponds in magnitude to a desired EMG signal.

6. The method of claim 1, wherein the secondary feature comprises a plurality of secondary features, wherein the frequency of the plurality of secondary features corresponds to a desired frequency of EMG signals.

7. A computer system for assisting a patient with neuro-muscular re-education therapy, the system comprising:
a mobile computing device, wherein the mobile computing device comprises a user interface and a game, wherein the game comprises a game feature and a secondary feature, wherein the secondary feature corresponds to a desired neuro-muscular re-education therapy movement; and
an EMG receiver coupleable to a patient's muscle, wherein the EMG receiver is communicatively coupled to the mobile computing device to transmit an EMG signal to the mobile computing device;
wherein the mobile computing device receives the EMG signal and modifies a characteristic of the game feature based on the EMG signal;
wherein the mobile computing device compares the modified characteristic of the game feature to the secondary feature to determine if the user's muscle performed the desired neuro-muscular re-education therapy movement.

8. The system of claim 7, wherein the EMG receiver is communicatively coupled to the mobile computing device by a wireless coupling.

9. The system of claim 7, wherein the EMG receiving comprises a plurality of EMG sensors, wherein the EMG sensors are coupled by a flexible coupling.

10. The system of claim 7, wherein the EMG receiver comprises a plurality of EMG sensors, wherein the EMG sensors are coupled by a rigid coupling.

11. The system of claim 7, wherein the desired neuro-muscular re-education therapy movement comprises flexing the user's muscle.

12. The system of claim 7, wherein the desired neuro-muscular re-education therapy movement comprises relaxing the user's muscle.

13. The system of claim 7, wherein an amplitude of the secondary feature corresponds to an amplitude of the desired neuro-muscular re-education therapy movement.

14. An apparatus for assisting a patient with neuro-muscular re-education therapy, the apparatus comprising:
a first EMG sensor comprising a first skin coupling portion for coupling to a user's skin at a user's muscle;
a second EMG sensor comprising a second skin coupling portion for coupling to the user's skin at the user's muscle;
a wireless transceiver for sending EMG signals received by the first and second EMG sensors to a mobile computing device comprising a game configured to assist the user with neuro-muscular re-education therapy;
a coupling between the first EMG sensor and the second EMG sensor.

15. The apparatus of claim 14, further comprising a barrier, an LED and an On/Off switch.

16. The apparatus of claim 14, further comprising an analog to digital converter and signal processing electronics.

17. The apparatus of claim 14, wherein the coupling is flexible to allow a user to position the first EMG sensor a user defined distance from the second EMG sensor.

18. The apparatus of claim 17, wherein the coupling is resilient and comprises a coil.

19. The apparatus of claim 14, wherein the coupling is rigid so that a distance between the first EMG sensor and the second EMG sensor is a predetermined distance.

20. The apparatus of claim 19, further comprising a third EMG sensor.
